(19) United States
(12) Patent Application Publication
(10) Pub. No.: US 2025/0098955 A1
(43) Pub. Date: Mar. 27, 2025

(54) SYSTEMS AND METHODS FOR ADJUSTING ILLUMINATION FOR OPTICAL IMAGING

(71) Applicant: Phoenix-Micron, Inc., Bend, OR (US)

(72) Inventor: Jonathan Roy Thorn, Bend, OR (US)

(73) Assignee: Phoenix-Micron, Inc., Bend, OR (US)

(21) Appl. No.: 18/892,995

(22) Filed: Sep. 23, 2024

Related U.S. Application Data
(60) Provisional application No. 63/540,272, filed on Sep. 25, 2023.

(51) Int. Cl. A61B 3/14

(52) U.S. Cl. CPC A61B 3/14

(57) ABSTRACT

The present disclosure is related to a system and method for adjusting the illumination intensity of an imaging device. The system includes an imaging camera including a light box. The outer surface of the light box includes a first set of magnets. The imaging camera includes a light guide configured to provide light to the light box. A focus assembly including a focus lens is disposed at an end of the light guide. An adjustable collar may be disposed around the focus assembly. The collar includes a second set of magnets disposed on a rear face of the main body. One or more set screws are disposed on the main body of the collar. The collar is configured to move along the length of the focus assembly and can be secured to a position on the focus assembly via the set screws.

### CROSS-REFERENCES TO RELATED APPLICATIONS

This application claims priority to and the benefit of U.S. Provisional Patent Application No. 63/540,272, filed Sep. 25, 2023, which is hereby incorporated by reference in its entirety for all intents and purposes.

### BACKGROUND OF THE INVENTION

Imaging devices rely on lenses to focus light for objects to be seen clearly. These imaging devices may include a light-tight box that is used to expose a photosensitive surface. The lens (e.g., focus lens) is used to focus and direct the light onto the photosensitive surface. The lens is an optical device that includes a curved material that allows light to pass through. Depending on the design, a camera lens includes one or more elements that both diverge and converge light to focus the light onto the photosensitive surface and re-assemble the light reflecting from the scene that has passed through the optics, resulting in an image.

The focus lens controls the amount of illumination entering the light box of an imaging camera. To adjust the amount or intensity of illumination, the position of the focus lens needs to be adjusted. However, imaging cameras fail to provide components that are modular to easily change the position of the focus lens. Therefore, adjusting the position of the focus lens essentially disables the system mechanically and optically. Additionally, adjusting the position of the focus lens may require a technician due to the complexity of the system.

Although imaging devices have been developed, there is a need in the art for improved systems and methods for modular imaging devices.

### SUMMARY OF THE INVENTION

The present disclosure relates to systems and methods for adjusting illumination of an imaging device for optical imaging. The system provides an adjustable stop collar disposed on a focus assembly to control the amount of light (e.g., focus) entering the light box of the imaging camera. This beneficially enables a user to tune the amount of light for specific imaging applications. For example, different ranges of illumination intensity can be provided based on the imaging application. The position of the collar can be manually adjusted to provide higher or lower illumination intensity for a desired application. Beneficially, the modular imaging device can reduce time between imaging and provide variable illumination intensities for different imaging applications. The system can reduce deconstruction, reconstruction, and manipulation of the components of the imaging system to change the illumination intensity.

Embodiments of the present disclosure include an imaging camera system. The imaging camera system includes a light box comprising an outer surface. A first set of magnets are disposed on the outer surface of the light box. The imaging camera system includes a light guide configured to provide light to the light box and a focus assembly including a focus lens disposed at a distal end of the light guide. A collar is disposed on the focus assembly. The collar comprises a main body comprising a front face and a rear face, a second set of magnets disposed on the rear face of the main body, and one or more set screws disposed on the main body. The collar is configured to slide along the focus assembly and can be secured to a position along a length of the focus assembly via the set screws.

Embodiments of the present disclosure include a method for adjusting an illumination intensity of an imaging system. The method includes providing an imaging camera comprising: a light box comprising an outer surface, wherein a first set of magnets are disposed on the outer surface of the light box; a light guide configured to provide light to the light box: a focus assembly including a focus lens disposed at a distal end of the light guide; a collar disposed on the focus assembly, wherein the collar comprises: a main body comprising a front face and a rear face; a second set of magnets disposed on the rear face of the main body; and one or more set screws disposed on the main body; wherein the collar is configured to slide along the focus assembly and can be secured to a position along the length of the focus assembly via the set screws. The method includes disengaging the set screws from the main body of the collar. The method includes determining a light intensity. The method includes moving the focus assembly to provide the determined light intensity. The method includes engaging the set screws on the main body to attach the collar to a position on the focus assembly. The method includes securing the second set of magnets on the collar to the first set of magnets on the outer surface of the light box.

Numerous benefits are achieved by way of the present disclosure over conventional eye imaging systems. For example, embodiments of the present disclosure provide a modular system for an imaging device that allows fine tuning of the illumination intensity. For example, the system allows for adjusting a position of a focus lens for a desired illumination intensity. As a result, the illumination intensity can be easily changed or adjusted without additional manipulation by an operator or breakdown of the entire imaging device. These and other embodiments of the disclosure, along with many of their advantages and features, are described in more detail in conjunction with the text below and attached figures.

### BRIEF DESCRIPTION OF THE DRAWINGS

FIG. 1 illustrates a focus assembly attached to a light box of an imaging device according to some embodiments.

FIGS. 2A and 2B illustrate adjusting the set screw to adjust the position of the collar on the focus assembly according to some embodiments.

FIG. 3 illustrates a focus assembly entering the light box of an imaging device according to some embodiments.

FIG. 4 illustrates a cutaway view of the focus assembly attached to a light box of an imaging device according to some embodiments.

FIGS. 5A-5C illustrate different illumination intensities of the focus assembly according to some embodiments.

FIG. 6 provides a flow diagram of a method of adjusting the illumination intensity of an imaging device according to some embodiments.

### DETAILED DESCRIPTION OF THE INVENTION

The present disclosure describes a number of embodiments related to a system and method for adjusting the illumination intensity of an imaging device (e.g., an eye imaging camera). In some embodiments, the present disclosure provides an imaging camera including a light box. The light box includes a through-hole for receiving a light guide configured to provide light to the light box. The outer surface of the light box includes a first set of magnets. In some embodiments, the magnets are adjacent the through-hole of the light box. A focus assembly including a focus lens is disposed at an end of the light guide. The focus assembly may include a lens to focus light from the light guide. For example, the focus lens of the focus assembly refracts light from the light guide into the light box. An adjustable collar may be disposed around the focus assembly. The collar includes a main body comprising a front face and a rear face. In some embodiments, the collar includes a second set of magnets disposed on the rear face of the main body. One or more set screws are disposed on the main body of the collar. The collar is configured to slide along the focus assembly and can be secured to a position along the length of the focus assembly via the set screws. The collar limits the extent the focus assembly extends into the light box to control the illumination intensity.

Conventional imaging devices comprise a focus lens that controls the amount of illumination entering the light box of an imaging camera. However, these imaging devices do not provide different ranges for illumination intensity for different applications. For example, imaging applications for detecting fluorescence in the eye may require high illumination intensity. On the other hand, imaging a highly reflective surface (e.g., retina of albino animal) may require low illumination intensity. In conventional imaging devices, the light guide and focus assembly that control illumination are located inside the imaging device and are not accessible. Therefore, the illumination intensity of the imaging device cannot be easily adjusted.

The system described herein provides an imaging device including a removable light guide (e.g., fiber guide) that attaches to the light box of the imaging camera. The light guide removably connects to the light box and is configured to provide light into the system. The focus assembly is disposed on a distal end of the light guide that connects to the light box. The focus assembly includes a focus lens and a Lyot stop. The system includes an adjustable collar that is disposed around the focus assembly. In some embodiments, the adjustable collar can slide along the focus assembly. For example, if the focus assembly is cylindrical, the adjustable collar can fit around the circumference of the focus assembly. The adjustable collar may include a fastener (e.g., set screws) that can secure the adjustable collar on a position on the focus assembly. Based on the position of the adjustable collar on the focus assembly, the distance the focus assembly extends into the light box can be controlled. In this way, the intensity of light into the light box can be tuned. In some embodiments, the adjustable collar includes a one or more magnets that can attach to one or more corresponding magnets on the exterior of the light box. Advantageously, the adjustable collar can be positioned along different regions of the focus assembly to control illumination intensity. In some embodiments, the adjustable collar can be unsecured to allow the focus assembly to move within the light box to a desired position. The set crews can be tightened to secure the adjustable collar at the desired position to provide a selected illumination intensity.

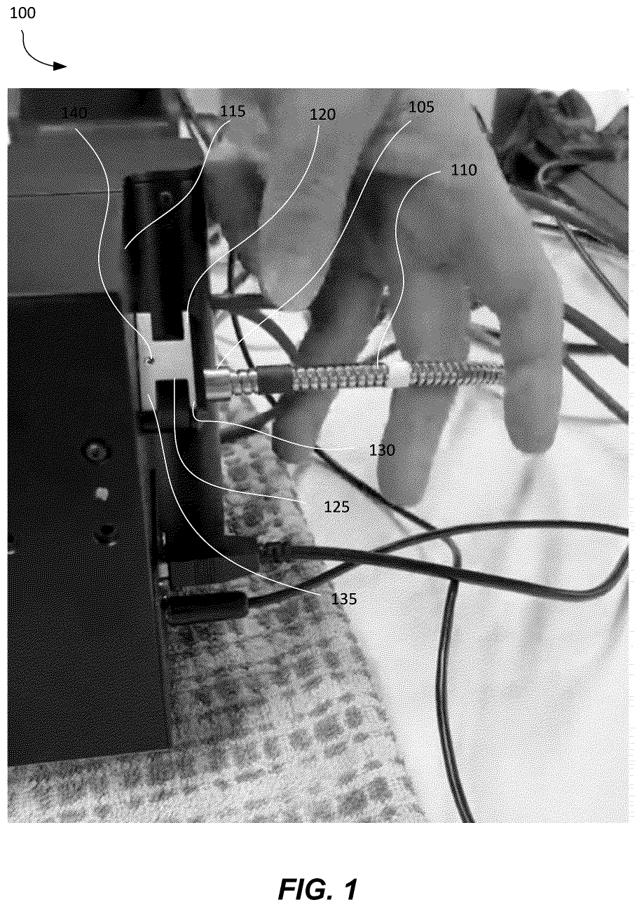

FIG. 1 shows a photograph of the system including an adjustable collar attached to a focus assembly. The focus assembly 105 is disposed at a distal end of a light guide 110. The light guide 110 is used to direct light from a light source (e.g., light emitting diode). In some embodiments, the light guide 110 is a fiber optic cable. Light that is provided to the light guide 110 is trapped within the light guide due to total internal reflection. The light that remains inside the light guide 110 can be provided to a light box 115. The light that is generated by a high intensity light source such as a lamp or bulb can held in the light box 115.

The system 100 includes a collar 120 disposed on the focus assembly 105. The collar 120 may be removable attached to the focus assembly 105. The collar 120 includes a main body 125. The main body 125 has three sides and is substantially triangular. In alternative embodiments, the main body 125 can have three or more sides. The main body 125 includes a front face 130 and a rear face 135. The front face 130 may be thicker than the rear face 135. The main body 125 may include a fastener 140. In some embodiments, the front face 130 and the rear face 135 comprise the same shape and are separated by the main body. In some embodiments, the fastener 140 can be set screws. The fastener 140 can be utilized to secure the collar 120 to the focus assembly 105. In some embodiments, the collar 120 can be positioned on the focus assembly 105 such that the illumination can be tuned to a desired intensity.

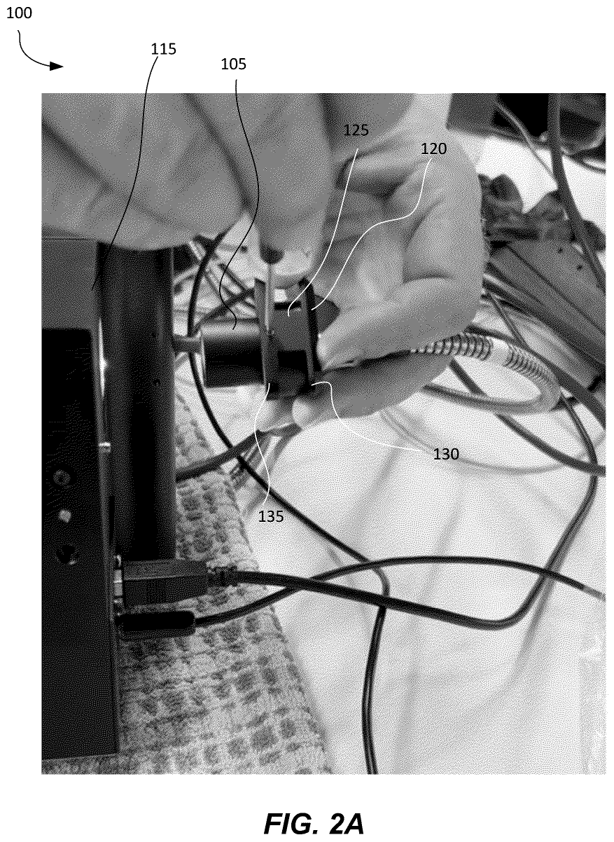

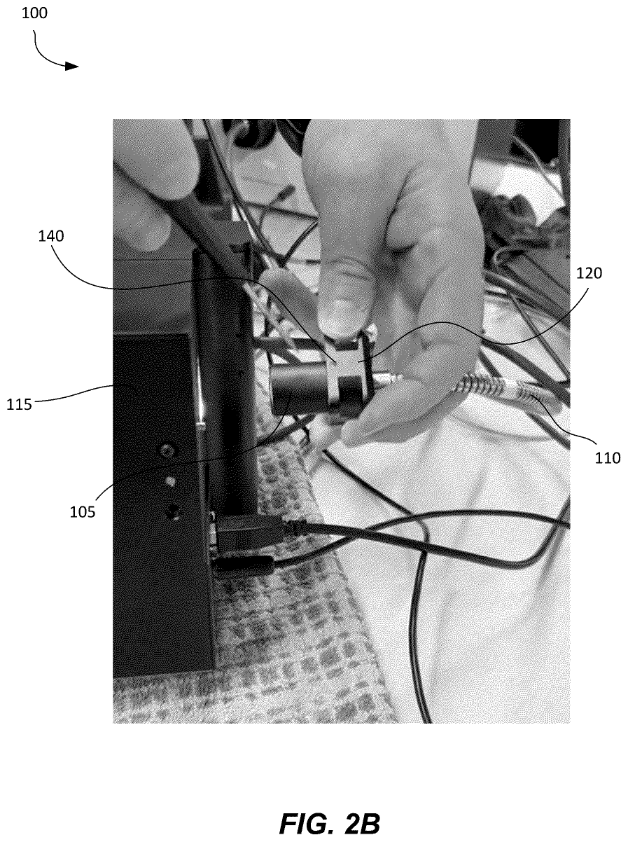

As shown in FIGS. 2A and 2B, the fastener 140 can be disengaged such that the collar 120 can be moved along the length of the focus assembly 105. For example, the set screws can be disengaged from the main body 125 such that the collar 120 can be moved along the focus assembly. In some embodiments, the collar 120 can be twisted to disengage the second set of magnets from the first set of magnets. In some embodiments, a tool can be used to disengage the set screws from the collar 120. In this way, the collar 120 can be moved along the length of the focus assembly 105. The intensity of light into the light box 115 can be controlled by adjusting the position of the collar 120. The collar 120 can move along different regions of the focus assembly 105 to control illumination intensity. Subsequently, the set screws can be tightened to secure the collar 120 at the desired position to provide a selected illumination intensity.

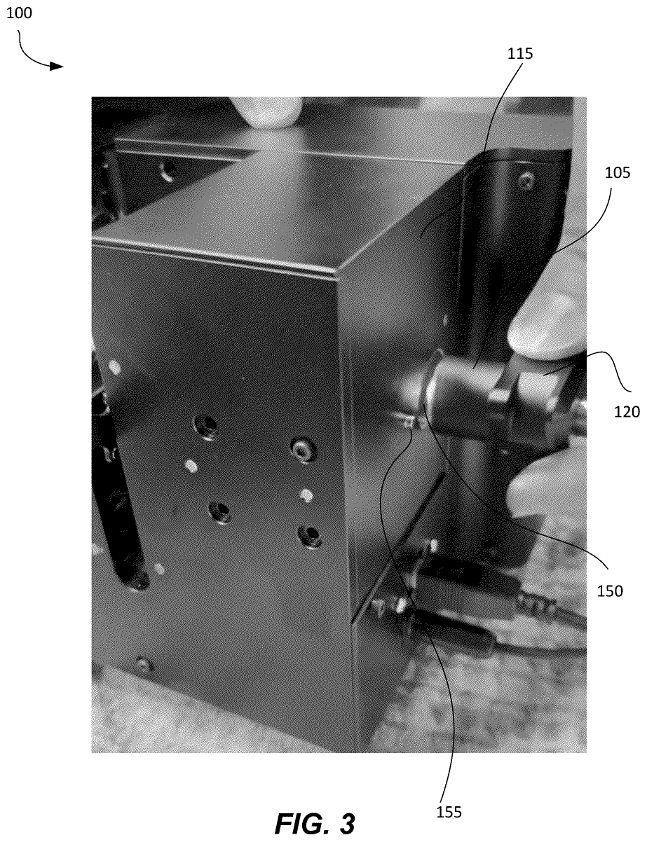

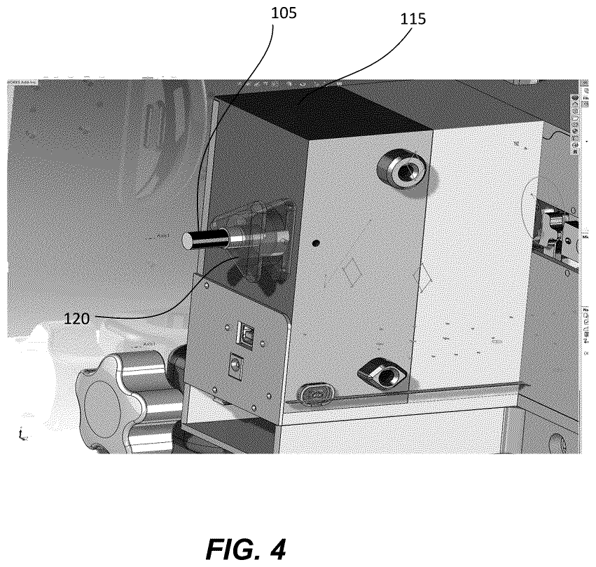

FIG. 3 shows the light guide 110 including the focus assembly 105 entering the light box 115. The focus assembly 105 can be inserted into the through-hole 150 of the light box 115. The collar 120 is positioned on the focus assembly 105 such that a length of the focus assembly 105 is within the light box 115. Based on the position of the collar 120 on the focus assembly 105, the distance the focus assembly 105 extends into the light box 115 can be controlled. In this way, the intensity of light into the light box 115 can be controlled. The collar 120 can be moved along a length of the focus assembly 105 to adjust the light intensity. For example, when the focus assembly 105 is received in a through-hole of the light box 115, the focus assembly 105 can be axially translated within the light box 115 to determine the light intensity and the collar 120 can be secured to the focus assembly to fix the location of the focus assembly within the light box 115. In some embodiments, the light box 115 includes a one or more magnets 155. The collar 120 may be magnetic or may include magnets that can attach to one or more magnets 155 on the exterior of the light box 115. FIG. 4 shows a cutaway view of the focus assembly 105 attached to the light box 115. The focus assembly 105 extends into the light box 115 to the extent of the collar 120. The collar 120 includes magnets that secure the focus assembly 105 to the light box 115.

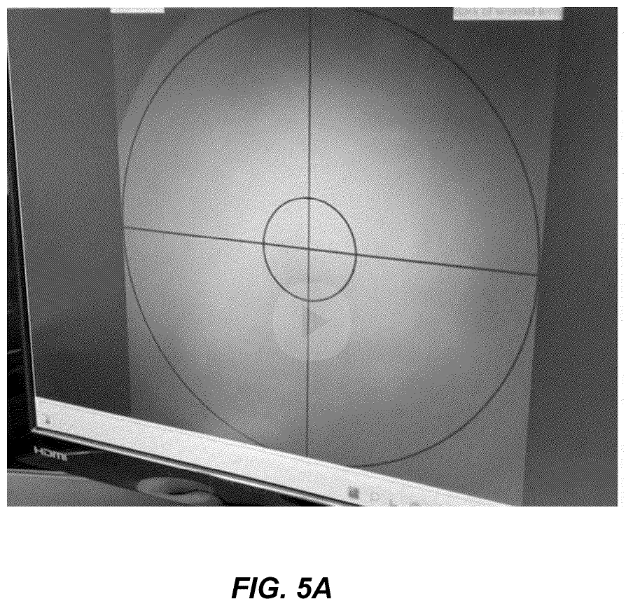

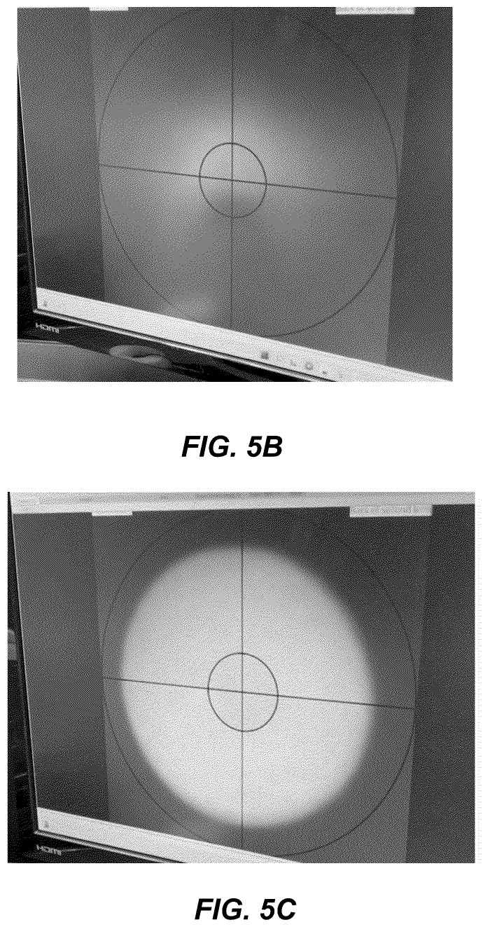

FIG. 5A to 5C show various illumination intensities of the focus assembly. The stop collar disposed on the focus assembly can tune the intensity of light entering the light box of the imaging camera. This beneficially enables a user to tune the amount of light for specific imaging applications. FIG. 5A shows the illumination intensity when the focus lens of the focus assembly is in a defocused state. In the defocused state, the focus assembly provides a low light intensity. As discussed herein, the collar can be fixed at a location on the focus assembly to provide the low intensity illumination shown in FIG. 5A. FIG. 5B shows a lower light intensity than FIG. 5A. In instances that require imaging of a highly reflective surface (e.g., retina of albino animal), the focus assembly can provide low illumination intensity. FIG. 5C shows the illumination intensity when the focus lens of the focus assembly is in a focused state. In the focused state, the focus assembly provides a high light intensity.

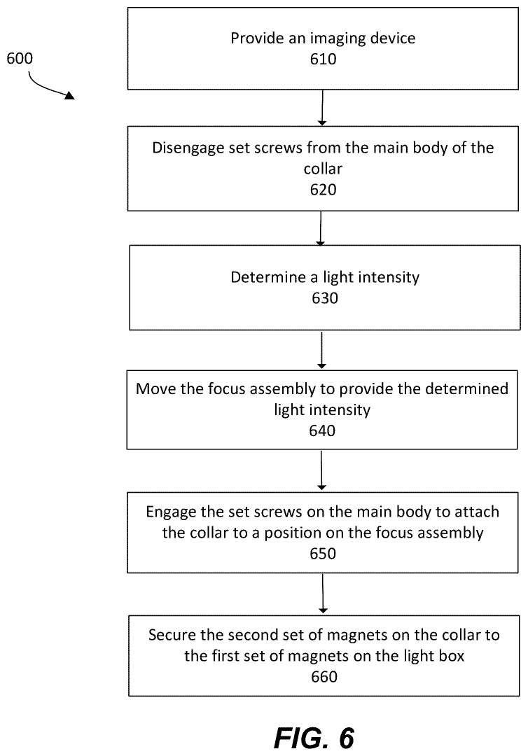

FIG. 6 provides a flow diagram of a method for adjusting the illumination intensity of an imaging device. The method 600 includes providing an imaging device 610. The imaging device includes a light box comprising an outer surface, wherein a first set of magnets are disposed on the outer surface of the light box. A light guide is configured to provide light to the light box. The light guide includes a focus assembly disposed at distal end of the light guide. A collar is disposed on the focus assembly. The collar includes a main body comprising a front surface and a rear surface; a second set of magnets disposed on the rear face of the main body; and one or more set screws disposed on the main body. The collar is configured to slide along the focus assembly and can be secured to a position along the length of the focus assembly via the set screws.

The method 600 includes disengaging the set screws from the main body of the collar 620. For example, an external tool may be used to disengage the set screws from the main body of the collar. In this way, the collar can move along the length of the focus assembly. The method 600 includes determining a light intensity 630. For example, a high or low light intensity can be selected based on the imaging application. In some embodiments, the focus assembly can be defocused to provide low light intensity or the focus assembly can be focused to provide high light intensity. The method 600 includes moving the focus assembly to provide the determined light intensity 640.

The method 600 includes engaging the set screws on the main body to attach the collar to a position on the focus assembly 650. The collar prevents the focus assembly from extending into the light box of the imaging camera past a given distance based on the location of the collar on the focus assembly. The method 600 includes securing the second set of magnets on the collar to the first set of magnets on the outer surface of the light box 660. The method may include capturing an image using the imaging device after the collar is secured to the light box.

It should be appreciated that the specific steps illustrated in FIG. 6 provide a particular method of imaging an eye according to some embodiments. Other sequences of steps may also be performed according to alternative embodiments. For example, alternative embodiments of the present invention may perform the steps outlined above in a different order. Moreover, the individual steps illustrated in FIG. 6 may include multiple sub-steps that may be performed in various sequences as appropriate to the individual step. Furthermore, additional steps may be added or removed depending on the particular applications. One of ordinary skill in the art would recognize many variations, modifications, and alternatives.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It will be apparent to one of ordinary skill in the art that many modifications and variations are possible in view of the above teachings.

### Embodiments

Embodiment 1: An imaging camera system comprising: a light box comprising an outer surface, wherein a first set of magnets are disposed on the outer surface of the light box; a light guide configured to provide light to the light box: a focus assembly including a focus lens disposed at a distal end of the light guide; and a collar disposed on the focus assembly, wherein the collar comprises: a main body comprising a front face and a rear face; a second set of magnets disposed on the rear face of the main body; and one or more set screws disposed on the main body; wherein the collar is configured to slide along the focus assembly and can be secured to a position along a length of the focus assembly via the set screws.

Embodiment 2: The embodiment of any preceding or subsequent embodiment, wherein the light guide comprises a fiber optic cable.

Embodiment 3: The embodiment of any preceding or subsequent embodiment, wherein the focus lens of the focus assembly refracts light from the light guide into the light box.

Embodiment 4: The embodiment of any preceding or subsequent embodiment, wherein the first set of magnets on the outer surface of the light box are configured to attach to the second set of magnets on the rear face of the collar.

Embodiment 5: The embodiment of any preceding or subsequent embodiment, wherein the main body of the collar comprises at least three sides.

Embodiment 6: The embodiment of any preceding or subsequent embodiment, wherein the main body is substantially triangular.

Embodiment 7: The embodiment of any preceding or subsequent embodiment, wherein the set screws are disposed adjacent the rear face of the main body.

Embodiment 8: The embodiment of any preceding or subsequent embodiment, wherein the front face and the rear face comprise the same shape and are separated by the main body.

Embodiment 9: The embodiment of any preceding or subsequent embodiment, wherein the rear face of the main body is thicker than the front face.

Embodiment 10: A method for adjusting an illumination intensity of an imaging system, the method comprising: providing an imaging camera comprising: a light box comprising an outer surface, wherein a first set of magnets are disposed on the outer surface of the light box; a light guide configured to provide light to the light box: a focus assembly including a focus lens disposed at a distal end of the light guide; a collar disposed on the focus assembly, wherein the collar comprises: a main body comprising a front face and a rear face; a second set of magnets disposed on the rear face of the main body; and one or more set screws disposed on the main body; wherein the collar is configured to slide along the focus assembly and can be secured to a position along a length of the focus assembly via the set screws; disengaging the set screws from the main body of the collar; determining a light intensity; moving the focus assembly to provide the determined light intensity; and engaging the set screws on the main body to attach the collar to a position on the focus assembly; and securing the second set of magnets on the collar to the first set of magnets on the outer surface of the light box.

Embodiment 11: The embodiment of any preceding or subsequent embodiment, further comprising capturing an image.

Embodiment 12: The embodiment of any preceding or subsequent embodiment, further comprising twisting the collar to disengage the second set of magnets from the first set of magnets.

Embodiment 13: The embodiment of any preceding or subsequent embodiment, wherein moving the focus assembly comprises translating the focus assembly within the light box to determine the light intensity, wherein the focus assembly is received in a through-hole of the light box

Embodiment 14: The embodiment of any preceding or subsequent embodiment, further comprising capturing an image using the imaging camera after securing the collar to the light box.

Embodiment 15: The embodiment of any preceding or subsequent embodiment, wherein the imaging camera is an eye imaging camera.

What is claimed is:

1. An imaging camera system comprising:
a light box comprising an outer surface, wherein a first set of magnets are disposed on the outer surface of the light box;
a light guide configured to provide light to the light box:
a focus assembly including a focus lens disposed at a distal end of the light guide; and
a collar disposed on the focus assembly, wherein the collar comprises:
a main body comprising a front face and a rear face;
a second set of magnets disposed on the rear face of the main body; and
one or more set screws disposed on the main body;
wherein the collar is configured to slide along the focus assembly and can be secured to a position along a length of the focus assembly via the set screws.

2. The imaging camera system of claim 1, wherein the light guide comprises a fiber optic cable.

3. The imaging camera system of claim 1, wherein the focus lens of the focus assembly refracts light from the light guide into the light box.

4. The imaging camera system of claim 1, wherein the first set of magnets on the outer surface of the light box are configured to attach to the second set of magnets on the rear face of the collar.

5. The imaging camera system of claim 1, wherein the main body of the collar comprises at least three sides.

6. The imaging camera system of claim 1, wherein the main body is substantially triangular.

7. The imaging camera system of claim 1, wherein the set screws are disposed adjacent the rear face of the main body.

8. The imaging camera system of claim 1, wherein the front face and the rear face comprise the same shape and are separated by the main body.

9. The imaging camera system of claim 1, wherein the rear face of the main body is thicker than the front face.

10. A method for adjusting an illumination intensity of an imaging system, the method comprising:
providing an imaging camera comprising:
a light box comprising an outer surface, wherein a first set of magnets are disposed on the outer surface of the light box;
a light guide configured to provide light to the light box:
a focus assembly including a focus lens disposed at a distal end of the light guide;
a collar disposed on the focus assembly, wherein the collar comprises:
a main body comprising a front face and a rear face;
a second set of magnets disposed on the rear face of the main body; and
one or more set screws disposed on the main body;
wherein the collar is configured to slide along the focus assembly and can be secured to a position along a length of the focus assembly via the set screws;
disengaging the set screws from the main body of the collar;
determining a light intensity;
moving the focus assembly to provide the determined light intensity; and
engaging the set screws on the main body to attach the collar to a position on the focus assembly; and
securing the second set of magnets on the collar to the first set of magnets on the outer surface of the light box.

11. The method of claim 10, further comprising capturing an image.

12. The method of claim 10, further comprising twisting the collar to disengage the second set of magnets from the first set of magnets.

13. The method of claim 12, wherein moving the focus assembly comprises translating the focus assembly within the light box to determine the light intensity, wherein the focus assembly is received in a through-hole of the light box.

14. The method of claim 10, further comprising capturing an image using the imaging camera after securing the collar to the light box.

15. The method of claim 10, wherein the imaging camera is an eye imaging camera.
